(19) United States
(12) Patent Application Publication
(10) Pub. No.: US 2021/0369454 A1
(43) Pub. Date: Dec. 2, 2021

(54) System and Method for Percutaneously Delivering a Tricuspid Valve

(71) Applicant: Synedcor LLC, Durham, NC (US)

(72) Inventors: Richard S. Stack, Chapel Hill, NC (US); Kevin W. Johnson, Durham, NC (US); William L. Athas, Chapel Hill, NC (US)

(21) Appl. No.: 17/173,158

(22) Filed: Feb. 10, 2021

Related U.S. Application Data
(60) Provisional application No. 62/972,586, filed on Feb. 10, 2020.

(51) Int. Cl. A61F 2/24

(52) U.S. Cl. CPC A61F 2/2427; A61F 2220/0016; A61F 2/2409

(57) ABSTRACT

According to a system and method for percutaneously delivering a replacement tricuspid valve to a heart, a wire is percutaneously introduced into the venous vasculature. A distal end of the wire is passed into a right atrium, through a tricuspid valve ring, and into a right ventricle, within which it is anchored to tissue of the right ventricle. A tricuspid valve delivery device carrying a replacement tricuspid valve is positioned over the wire, and a director is positioned over the wire proximally adjacent to, and in contact with, the tricuspid valve delivery device. The director and delivery device are pushed over the wire into the right atrium while traction is applied traction to the wire that is fixed within the right ventricle. The director is actively articulated during advancement, to articulate the replacement tricuspid valve on the valve delivery device within a tricuspid valve ring of the heart.

This application claims the benefit of U.S. Provisional Application No. 62/972,586, filed Feb. 10, 2020.

### BACKGROUND

Few minimally invasive techniques for treating the tricuspid valve are currently available. While desirable, the ability to percutaneously deliver a replacement tricuspid valve is a particular challenge that has not yet found a suitable solution, as the large proportions and stiffness of a delivery system carrying a tricuspid valve replacement render it difficult to maneuver to the target site.

This application describes a system and method for use in delivering a tricuspid valve delivery system carrying a tricuspid valve replacement device to the tricuspid valve annulus.

### BRIEF DESCRIPTION OF THE DRAWINGS

FIGS. 1A-2 show components of the disclosed system in which:

FIG. 1A is a perspective view of a redirector;

FIG. 1B is a side elevation view of the portion of the redirector encircled in FIG. 1A; and

FIG. 2 is a perspective view of an anchor wire device;

FIG. 3A is a perspective view of a first embodiment of an anchor wire device, in the retracted position;

FIG. 3B is similar to FIG. 3A but shows the anchor wire device in the extended position;

FIG. 3C is similar to FIG. 3B but has the screw housing removed to permit easy viewing of the surrounding features.

FIG. 4A is a perspective view of a second embodiment of an anchor wire device, in the retracted position;

FIG. 4B is similar to FIG. 4A but shows the anchor wire device in the extended position;

FIG. 4C is similar to FIG. 4B but has the screw housing removed to permit easy viewing of the surrounding features.

FIG. 5 is a schematic right ventricular AP view of the heart;

FIG. 6 shows the RV AP view of the heart shown in FIG. 5, with the right atrium and right ventricle cut and associated vasculature cut away to allow the step of positioning the director in the right ventricle to be seen;

FIG. 7 is a similar view to FIG. 6, and shows deployment of the anchor wire through the director;

FIG. 8 is a similar view to FIG. 7, and shows the step of anchoring the anchor wire to the septum of the right ventricle;

FIG. 9 is a close up view of the region of the director and anchor wire encircled in FIG. 8;

FIG. 10 is a similar view to FIG. 8, and shows the anchor wire anchored in the right ventricle after the removal of the director;

FIGS. 11 and 12 illustrates the steps of positioning the valve delivery balloon and valve onto the anchor wire and seating the proximal part of the delivery balloon and valve assembly at the distal opening of the director;

FIG. 13 is a similar view to FIG. 10 and shows advancement of the director and balloon/replacement valve assembly over the wire towards the right ventricle;

FIG. 14 is a similar view to FIG. 13 and illustrates use of the director to steer the balloon/replacement valve assembly at the tricuspid valve ring of the heart;

FIG. 15 is similar to FIG. 14 and shows deployment of the valve at the tricuspid valve ring;

FIG. 16 shows the replacement valve in place at the tricuspid valve ring following removal of the anchor wire and director.

### DETAILED DESCRIPTION

The system and method described below allow percutaneous delivery of a replacement valve using an access point in the venous vasculature, such as a femoral vein. In use the system facilitates movement of the replacement valve from the access point, through the inferior vena cava (IVC) to the right atrium (RA), allowing articulation of the assembly through the acute angle needed to properly orient the replacement valve within the native valve ring.

System

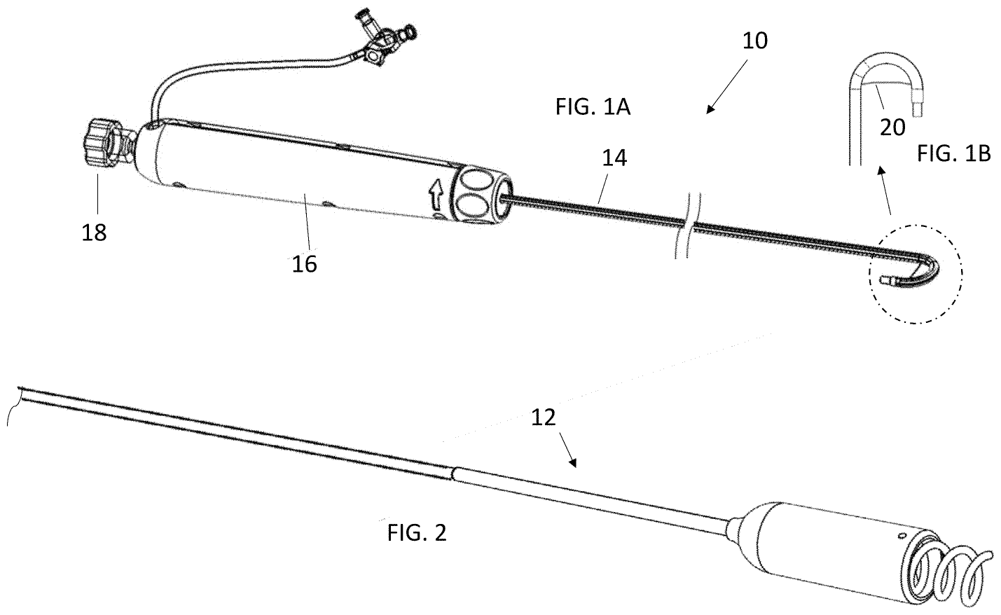

Referring to FIGS. 1A through 2, components of the system include a director 10 in the form of a steerable lumen device, and an anchor wire device 12 having a screw tip that can be engaged with the tissue of the right ventricle septum so that the director and replacement valve assembly can be directed to the target site.

In general, the director 10 possesses the ability to direct the replacement valve assembly through a significant articulation angle (as described below) from the IVC to the RA and into the valve ring, without buckling. While various configurations of steerable catheter may be used for the director, a preferred director 10 will have properties similar to those of the “LVR” described in co-pending U.S. application Ser. No. 16/578,379, incorporated herein by reference.

The director 10 includes an elongate catheter shaft 14 having a proximal handle 16 with a proximal access port 18 and a flush port. The shaft includes a lumen accessible via the access port 18. This lumen extends to the distal tip of the shaft.

The distal end of the shaft 14 is moveable between a generally straight position and an articulated position in which the distal end is formed into a curve, as shown in FIGS. 1A and 1B. The handle 16 includes actuators to actuate pull wires that run through the shaft, to bend the shaft and to actuate a return wire to return the distal end of the shaft to the generally straight configuration.

One of the pull wires 20 exits the sidewall of the shaft near the shaft's distal end, runs along the exterior of the shaft in a distal direction, and re-enters the shaft at the distal end of the shaft, while the other pull wire does not exit the shaft at the distal end. The dual pull wire configuration advantageously allows articulation to the desired curvature and locking of the articulation in that curvature despite high loads that could be experienced at the tip of the director during use.

The pull wire that remains inside the shaft (“internal pull wire”) helps maintain the patency of the shaft's lumen during articulation, preventing the shaft from buckling or kinking despite the large degree of articulation as would likely happen if the construction used only the external pull wire.

The external pull wire 20 functions as a locking mechanism to lock the shaft in its articulated orientation, preventing the curve from opening when forces are exerted against its distal tip.

Note that the terms “pull wire” and “wire” are not intended to mean that these elements must be formed of wire, as these terms are used more broadly in this application to represent any sort of tendon, cable, or other elongate element. Also, while the term “straight” is used to refer to the shape of the director distal portion in its non-articulated position, it should be pointed out that the catheter's inherent flexibility in the non-articulated position may cause it to bend under forces of gravity when held upright, or to curve when tracked over a curved cable or wire, or advanced into contact with another structure. The term “straight” thus should not be used to interpret this application or the corresponding claims as requiring that portion of the director shaft to hold a straight shape.

The pull wire and return wire configuration preferably provide for steering in two directions, with movement occurring along one plane P1 between straight and curved positions. Other embodiments can be configured with additional directions of movement if desired.

Turning now to a discussion of the anchor wire device 12, this device comprises an elongate wire of sufficient length to extend from the right femoral vein, through the IVC, into the RA and through the tricuspid valve (TV) into the right ventricle (RV). Its distal end includes an anchor used to releasably anchor the distal end of the wire in the right ventricle with the wire extending through the tricuspid valve. As one example, the anchor may be releasably engageable with the right ventricular septum (RV septum).

The first and second embodiments described below include screw anchors for this purpose, but it should be appreciated that other types of anchors may be used, including hooks, expanding collets, clips, lengths of suture passed through the tissue and secured, and others not listed here. In an alternative configuration, rather than anchoring to tissue of the RV, the wire could utilize a balloon or other expandable anchor that could be expanded in a pulmonary artery branch to anchor the wire.

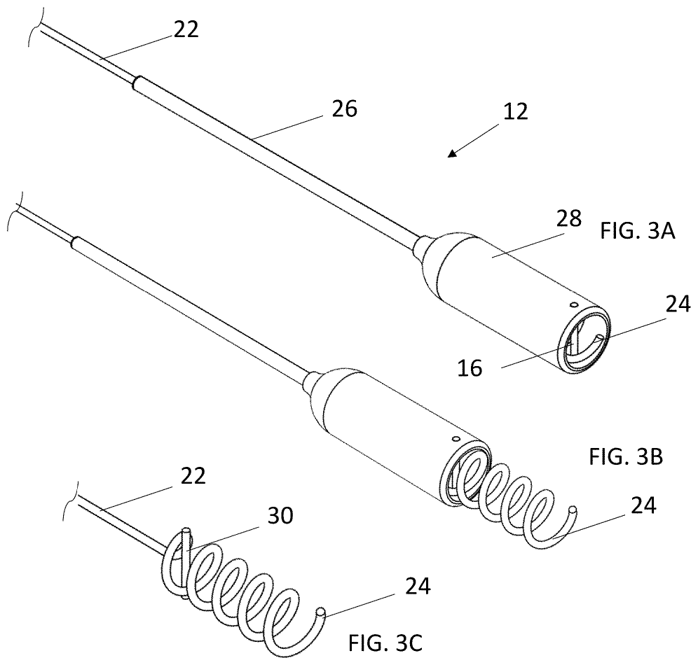

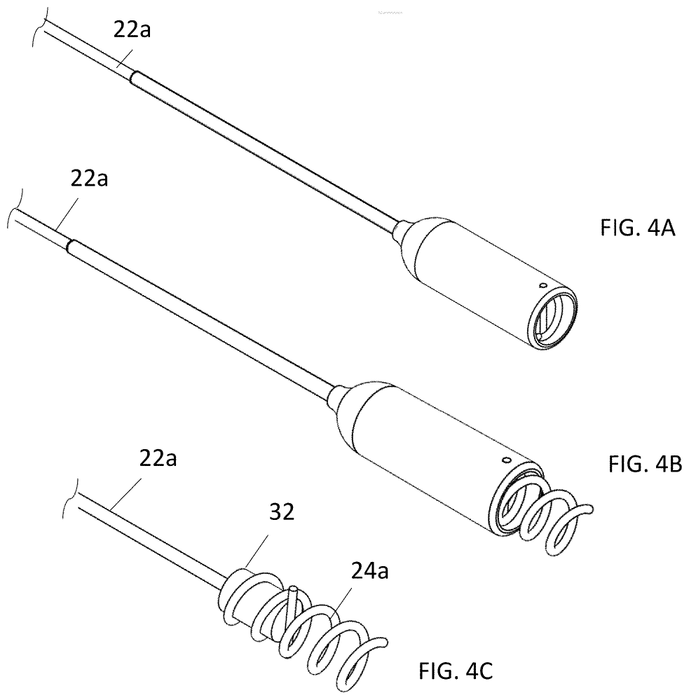

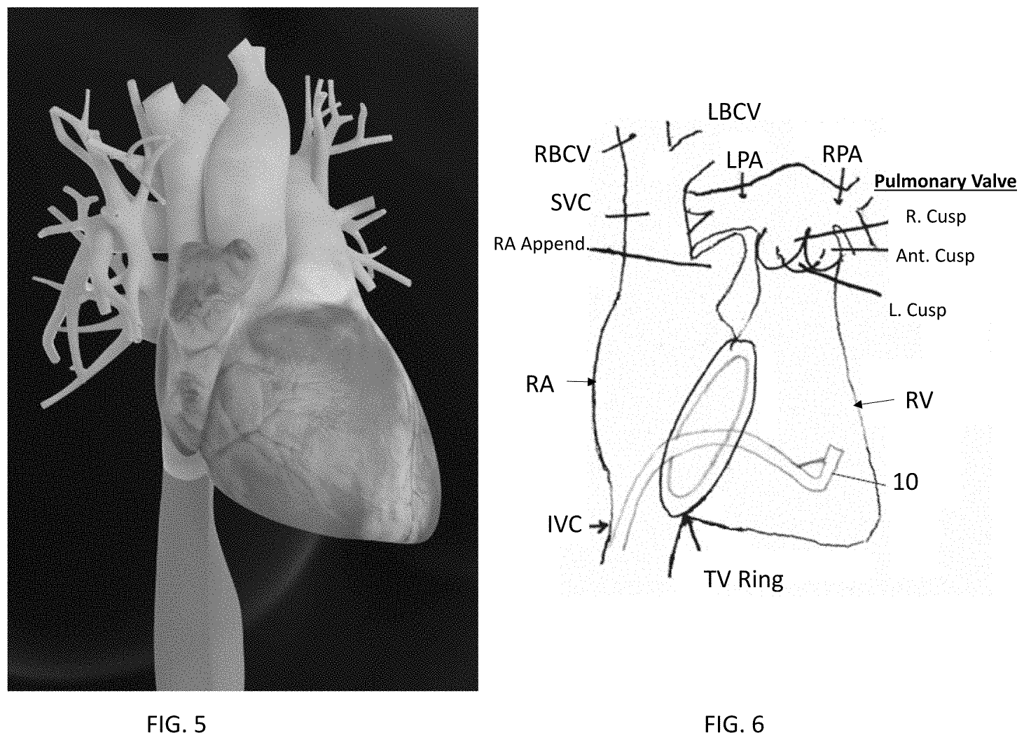

Referring to FIGS. 3A-3C, the first embodiment of the anchor wire device includes a wire 22 and anchor 24 at the distal end of the wire. In the embodiments shown, the anchor 24 is a screw which may be formed of helical member that will advance into the tissue when rotated while being pressed against the tissue. The screw may be a shape memory (e.g. Nitinol) wire or filament heat set into the helical shape. In the FIG. 3A-3C embodiment, the screw anchor 24 and wire 22 may be a single length of material. A torquer (not shown) can be attached to the proximal end of the wire 22, outside the body, and rotated to advance the screw 24 into tissue.

A tube 26 is slidably positioned over the anchor 24 and includes a distal housing 28. Tube 26 may be formed of a length of hypotube. Housing 28 is proportioned such that when it is advanced to the distal end of the anchor 24, the anchor 24 is positioned within the housing. Withdrawing the tube 26 relative to the anchor in a proximal direction exposes at least a portion of the anchor 24 so that it can be engaged with the tissue. A pin 30 extends across the lumen of the housing between windings of the screw coil helps guide the coil anchor out of the housing and serves as a stop to limit the amount of the anchor 24 that can extend from the distal end of the housing.

The tube 26 preferably, but optionally, has a diameter of approximately 0.035 inches or less, allowing commercially available valve delivery systems (conventionally designed to pass over 0.035″ guidewires) to pass over it.

The second embodiment, shown in FIGS. 4A-4C is largely similar to the first embodiment, but differs in that the anchor 24a and wire 22a are not integral as with the first embodiment, but are separate pieces welded together, such as by using a base 32 that they are each welded to.

The wire additionally has a backstop that, as will be understood from reviewing the sequence of steps depicted in the drawings, is used to aid in deployment of the valve at the tricuspid valve annulus.

Method

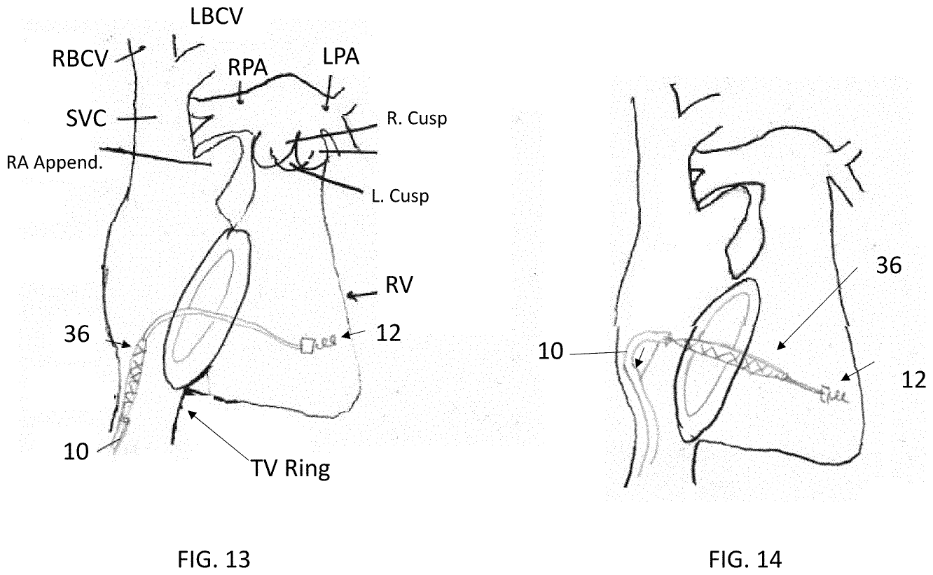

A method of delivering a replacement tricuspid valve will next be described with reference to FIGS. 6 through 16.

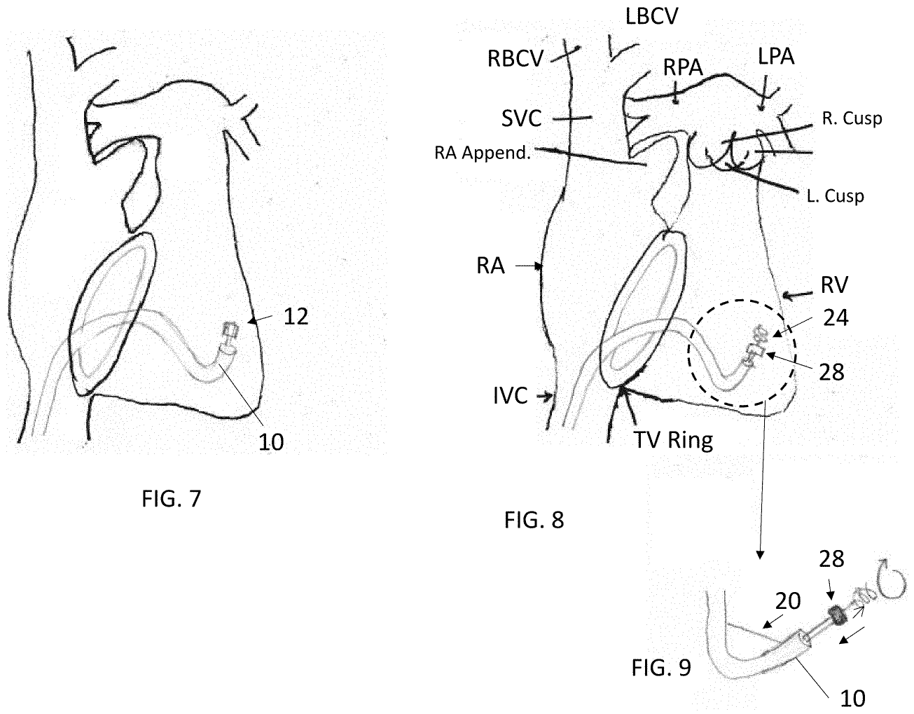

The director 10 is percutaneously introduced into the left or right femoral vein via a femoral sheath, and advanced over a guidewire to the IVC and RA, and then through the TV to the RV. FIG. 6. The anchor wire device 12 is advanced through the director 10 until it extends from the open end of the director's lumen. FIG. 7.

Referring to FIGS. 8 and 9, the housing 28 is withdrawn from the anchor 24 to expose the anchor coil. The anchor is positioned in contact with tissue of the RV septum. The anchor 24 is rotated by rotating its shaft (e.g. using a torquer attached to the wire shaft 22 outside the body) while pushing against the tube 26 to press the coiled anchor distally as it is being rotated, allowing it to become fixed within the tissue.

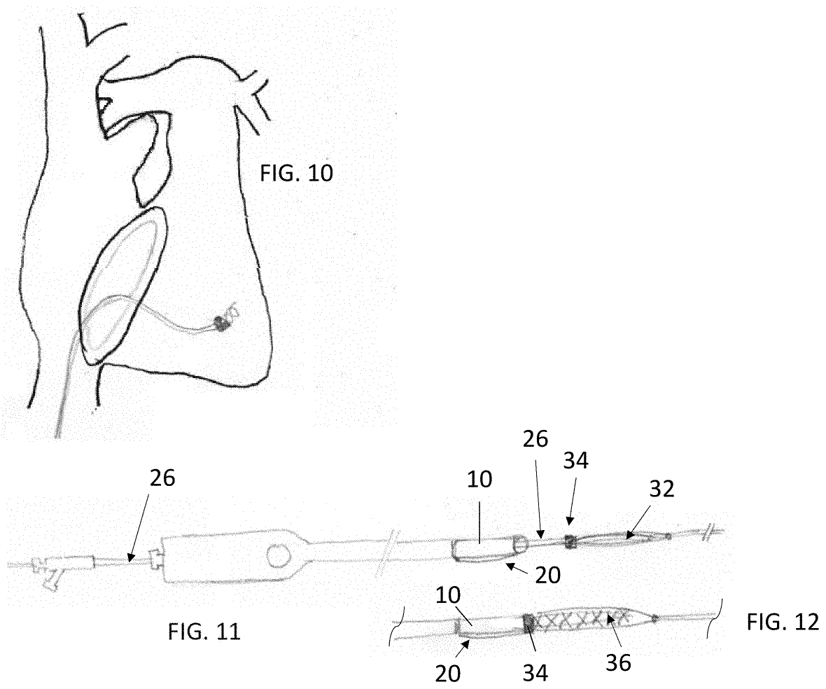

The director 10 is removed from the body, leaving the anchor wire 24 fixed within the RV. FIG. 10.

Outside the body, a tricuspid valve delivery system including a valve deployment balloon 32 is threaded onto the tube 26 and positioned extending through the director 10. With the delivery system positioned with the valve deployment balloon on the tube 26 and distal to the distal end of the director 10. A backstop 34 for the valve is connected (or pre-connected), such as by crimping, to the delivery system. FIG. 11. The replacement tricuspid valve 36 is crimped onto the balloon 32. The director 10 is advanced over the tube 26 until its distal lumen engages with the crimp 34 (FIG. 12), closing the gap between the director 10 and crimp 34 that is shown in FIG. 11.

The assembled system is introduced into the femoral sheath and advanced to the RA over the tube 26, by pushing on the valve delivery system and/or director 10 and pulling on the wire 22 that is fixed to the RV septum. FIG. 14.

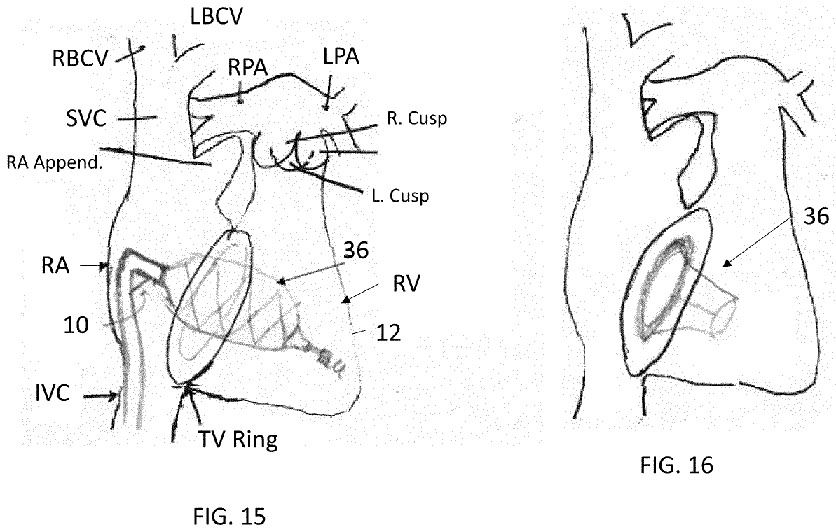

Traction is applied to the wire while, as shown in FIG. 14, the director 10 is steered to articulate the valve into the TV ring, and to center the valve within the ring. The balloon is expanded to deploy the valve (FIG. 15). The balloon is subsequently deflated, and the anchor is disengaged from the RV such as by rotating it to unscrew the coil from the tissue. The director 10, anchor wire 12 and delivery system are removed from the body, leaving the valve in place as shown in FIG. 16.

In an alternative method, the delivery system can be introduced via any superior venous access site (e.g. a brachiocephalic vein or internal jugular vein to the superior vena cava), so that it can approach the tricuspid valve from above. In further alternatives, the method may employ two systems, which could be introduced simultaneously or sequentially. In such alternatives, one system is deployed from below (e.g. a femoral vein) and one from a superior access site, or both may be deployed from superior access sites or inferior access sites, in order to deploy: an annuloplasty device, valve ring (to be used as a valve “docking station), valve (either balloon expandable or self-expanding), two tandem valves, any form of valve repair device.

In other alternative embodiments, the director could be used alone without an anchoring wire in order to precisely position tricuspid therapeutic devices for deployment.

All prior patents and applications referred to herein, including for purposes of priority, are incorporated herein by reference.

We claim:

1. A method of percutaneously delivering a replacement tricuspid valve to a heart, comprising the steps of:
percutaneously introducing a wire into a venous vasculature, and passing a distal end of the wire into a right atrium, through a tricuspid valve ring, and into a right ventricle;
anchoring a distal end of the wire to tissue of the right ventricle;
positioning a tricuspid valve delivery device over the wire, the tricuspid valve delivery device carrying a replacement tricuspid valve;
positioning a director over the wire, proximally adjacent to the tricuspid valve delivery device;
advancing the director and the tricuspid valve delivery device over the wire into the right atrium while applying traction to the wire;
actively articulating the director while applying traction to the wire, to articulate the replacement tricuspid valve on the valve delivery device within a tricuspid valve ring of the heart;
using the valve delivery device, expanding the replacement tricuspid valve within the ring; and
removing the director and the wire from the heart.

2. The method of claim 1, wherein anchoring the wire includes screwing an anchor on the wire into tissue of the right atrium.

3. The method of claim 1, wherein anchoring the wire includes clipping an anchor on the wire into tissue of the right atrium.

4. The method of claim 1, wherein actively articulating the director includes increasing tension on at least one pull wire in the director.

5. The method of claim 5, wherein actively articulating the director includes increasing tension on at least one external pull wire, the external pull wire extending internally through a proximal portion of a shaft of the director and extending longitudinally along an exterior of a distal portion of the shaft.

5. The method of claim 1, wherein introducing a wire into a venous vasculature comprises introducing the wire via a femoral vein.

6. The method of claim 1, wherein introducing a wire into a venous vasculature comprises introducing the wire via a venous access point superior to the heart.

7. A system for delivering a replacement tricuspid valve to a heart, the system comprising:
an anchor wire device comprising an elongate wire having an anchor on its distal end, the anchor releasable engageable with tissue within a heart, the wire of sufficient length that when the anchor is engaged with tissue of a septum in a right ventricle, the wire extends in an operative position from the right ventricle, through a native tricuspid valve ring into a right atrium and out the venous vasculature to a percutaneous access point;
a director advanceable over the anchor wire device, the director including a steerable distal end, and at least one actuator manipulatable by a user to actively steer the distal end; and
a tricuspid valve delivery system having a tricuspid replacement valve thereon;
the tricuspid valve delivery system and director positionable on the wire when the wire is in the operative position, with the tricuspid valve delivery system and director in a cooperative arrangement in which the delivery system is distally adjacent to and in contact with the director,
the director in the cooperative arrangement actively steerable using the actuator to steer the valve delivery system across the tricuspid valve ring and into an orientation perpendicular to the plane of the valve ring.

8. The system of claim 7, wherein the anchor is a screw anchor.

9. The system of claim 8, wherein the screw anchor is a coil.

10. The system of claim 7 wherein the anchor is a clip.

11. The system of claim 10, wherein the clip includes at least two members having a first, clipping, position in which a tissue-contacting parts of the members are spaced by a first distance, and a second, releasing, position in which the tissue-contacting parts are spaced by a second distance larger than the first distance.

12. The system of claim 7 wherein the director includes at least one pull wire, wherein actuation of the actuator modifies tension on the pull wire.

13. The system of claim 12, wherein the pull wire is an external pull wire, the external pull wire extending internally through a proximal portion of a shaft of the director and extending longitudinally along an exterior of a distal portion of the shaft.
